(19) United States
(12) Patent Application Publication
(10) Pub. No.: US 2011/0033932 A1
(43) Pub. Date: Feb. 10, 2011

(54) SUBSTRATE FOR CELL ADHESION OR CULTURE AND METHOD FOR PRODUCING THE SAME

(75) Inventors: Atsushi Kira, Chigasaki-shi (JP); Kou Fuwa, Chigasaki-shi (JP)

Correspondence Address: GROSSMAN, TUCKER, PERREAULT & PFLEGER, PLLC, 55 SOUTH COMMERICAL STREET, MANCHESTER, NH, 03101, US

(73) Assignee: ULVAC, INC., Chigasaki-shi (JP)

(21) Appl. No.: 12/744,135

(22) PCT Filed: Nov. 21, 2008
(86) PCT No.: PCT/JP2008/071243

§ 371 Date: Oct. 28, 2010

(30) Foreign Application Priority Data
Nov. 21, 2007 (JP) 2007-302246

(51) Int. Cl. C12N 5/00; G03F 7/20

(52) U.S. Cl. 435/395; 430/320

(57) ABSTRACT

A substrate for cell adhesion or culture of the invention comprises: a base material; a cell adhesion layer arranged to cover a predetermined region on this base material; and a non-cell adhesion layer arranged on the base material to cover a region other than the predetermined region. An exposed surface of the cell adhesion layer is a cell adhesion surface. The non-cell adhesion layer has a light transmission characteristic, and an exposed surface of the non-cell adhesion layer is a non-cell adhesion surface.

### TECHNICAL FIELD

The present invention relates to a substrate for cell adhesion or culture, on the surface of which cells can be arranged highly precisely at a high density, and with which the function of cells can be analyzed with high accuracy, and a method for producing the same.

Priority is claimed on Japanese Patent Application No. 2007-302246, filed Nov. 21, 2007, the content of which is incorporated herein by reference.

### BACKGROUND ART

Techniques to keep animal or plant cells alive are important to analyze functions of cells, interactions between cells and chemical agents, and the like. Such analyses contribute to the elucidation of life phenomena, the production of useful substances, and assays for analyzing responses of cells against physiological activities or toxicity of chemical agents.

Some kinds of cells, in particular animal cells, have adhesion dependency which is a manner of growth to survive by adhering to some material. For this reason, it is difficult for them to survive for a long time in a floating state in vitro. Accordingly, techniques to adhere cells onto a substrate while keeping them alive are important whether or not they need to be cultured. So far, various methods have been examined, and there is a disclosed method in which cells are adhered onto a substrate coated with an adhesive protein such as a collagen and a fibronectin. In general, such analyses often use cultured cells.

In conventional approaches, a plurality of cells are collectively analyzed as a group to obtain their average data, and the average value is used for analyses as if it were the characteristic of each cell. However, in fact, cells are seldom synchronized in a group in terms of the cell cycle and a daily rhythm called the circadian rhythm, and cells do express genes and proteins according to individually different cycles. For this reason, responses against stimuli of chemical agents or the like involve the problem of fluctuation. Therefore, in order to solve this problem, approaches such as a synchronous culture are being developed and utilized. In this approach, in order to use cells at the same stages at all times, it is necessary to continuously supply such cells. Thus, it takes much time and labor consuming, which becomes a problem of the assay methods using cells.

Therefore, there is a vigorous movement for a more highly accurate analysis, to culture each single cell and then to measure the data of each cell within a cell group.

For example, there have been proposed; a technique in which a specific single cell is exclusively selected and the single cell is cultured as a cell line, a technique in which the environmental condition of a cell medium is controlled and the concentration of cells in the container is controlled at a constant level for the observation of cells, and a technique in which the culture and the observation are performed while specifying cells which interact with each other (refer to Patent Document 1).

In addition, recently, for the analysis of cells under the above-mentioned purposes, the analysis method is required to be more efficient with diverse functions, and there is a demand for the development of a technique to arrange and adhere cells onto an ultra small area of a substrate. Such an adhesion technique is expected to greatly contribute to the progress of, for example, artificial internal organs, biosensors, and bioreactors which utilize cultured cells. Therefore, there is a proposed method for the arrangement of cells on an ultra small area of a substrate, wherein patterning ultra small areas having different cell adhesion properties, and adhering cells selectively onto the area having a higher cell adhesion property (for example, refer to Patent Document 2).

Patent Document 1: Japanese Unexamined Patent Application, First Publication No. 2004-81085

Patent Document 2: Japanese Unexamined Patent Application, First Publication No. H05-176753

### DISCLOSURE OF INVENTION

### Problems to be Solved by the Invention

However, conventionally proposed methods such as the methods described in Patent Documents 1 and 2 can not be said to be suitable for highly precise arrangement of cells in a single layer. Therefore, there is a demand for the development of a new type of substrate on which cells can be arranged highly precisely at a high density. In particular, optical methods such as a microscope or microscope in combination with a laser are effective as a method for analyzing cells. For this reason, transparent substrates which are useful for the analysis with such optical methods are desired to be developed.

The present invention was made to address the above-mentioned situations with an object of providing a substrate for cell adhesion or culture, on which cells can be adhered and arranged highly precisely at a high density, and with which the function of cells can be analyzed with high accuracy.

### Means for Solving the Problems

In order to solve the above-mentioned problems and to achieve the concerned object, the present invention employs the following.

(1) A substrate for cell adhesion or culture of the present invention comprises: a base material; a cell adhesion layer arranged to cover a predetermined region on the base material; and a non-cell adhesion layer arranged on the base material to cover a region other than the predetermined region; wherein an exposed surface of the cell adhesion layer is a cell adhesion surface; the non-cell adhesion layer has a light transmission characteristic; and an exposed surface of the non-cell adhesion layer is a non-cell adhesion surface.

(2) It is preferable that the non-cell adhesion layer contains a fluoride-containing compound represented by the following general formula (1):

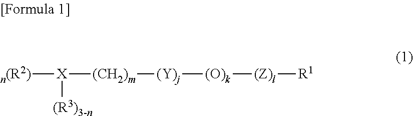

(wherein, R¹ represents a linear or branched perfluoroalkyl group or perfluoroalkylether group of 1 to 16 carbon atoms; R² represents a hydroxyl group, or an atom or group which can be substituted by a hydroxyl group; R³ represents a hydrogen atom or a monovalent hydrocarbon group; X represents a silicon atom or a phosphorus atom; Y is a group represented by —NH—C(═O)— or a carbonyl group; Z represents an ethyleneoxy group having a hydrogen atom substituted by an alkyl group or alkyloxyalkyl group, one or more hydrogen atom(s) of which can be substituted by fluorine atom(s); j and k represent 0 or 1; 1 and m represent an integer of 0 or larger; n represents 1, 2, or 3; if n represents 2 or 3, n pieces of R² may be the same as or different from each other; if n represents 1, two pieces of R³ may be the same as or different from each other; and if 1 represents an integer of 2 or larger, 1 pieces of Z may be the same as or different from each other).

(3) It is preferable that the fluoride-containing compound represented by the general formula (1) is a compound represented by the following general formula (2):

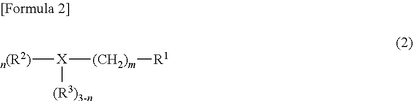

(wherein, R¹, R², R³, X, m, and n have the same meanings as those mentioned above).

(4) It is preferable that a level difference is provided between the predetermined region covered by the cell adhesion layer and the region covered by the non-cell adhesion layer on the base material.

(5) It is preferable that a height of the level difference is 0.01 to 100 μm.

(6) It is preferable that a thickness of the non-cell adhesion layer is 5 to 200 nm

(7) It is preferable that a plurality of the cell adhesion surfaces having dimensions of 2.0×10⁻¹¹ to 4.0×10⁻⁸ m² are connected by the cell adhesion surface in a belt-like shape having a width of 1×10⁻⁶ to 3×10⁻⁵ m.

(8) A method for producing the substrate for cell adhesion or culture according to (1), comprises covering the surface of a base material with a non-cell adhesion layer; removing a specific site of this non-cell adhesion layer to expose the surface of the base material; and covering the thus exposed surface of the base material with a cell adhesion layer.

(9) It is preferable that a surface of the base material is exposed by: covering the surface of the base material with a non-cell adhesion layer; making a pattern on this non-cell adhesion layer with a photosensitive resin; and removing the specific site of the non-cell adhesion layer by etching with use of the pattern of the photosensitive resin as a mask, and subsequently removing the photosensitive resin.

(10) It is preferable that a surface of the base material is exposed by: making a pattern on the surface of the base material with a photosensitive resin; covering the surface of the base material and the photosensitive resin with a non-cell adhesion layer; and removing the non-cell adhesion layer on a site of the non-cell adhesion layer covering the photosensitive resin together with the photosensitive resin covered by this non-cell adhesion layer.

(11) It is preferable that the method further comprises forming a level difference in the surface of the base material; and covering the surface of the base material with the non-cell adhesion layer after forming the level difference.

(12) It is preferable that the etching is performed with use of a plasma generated from a gas selected from the group consisting of oxygen, argon, and chlorine.

### EFFECT OF THE INVENTION

According to the substrate for cell adhesion or culture described in (1), cells can be adhered and arranged highly precisely at a high density on the substrate, and the function of cells can be analyzed with high accuracy. Moreover, various analyses and material productions utilizing the function of cells can be performed with high efficiency.

### BRIEF DESCRIPTION OF THE DRAWINGS

FIG. 1A is an enlarged plan view showing an example of a substrate according to a first embodiment of the present invention.

FIG. 1B is a longitudinal cross-sectional view taken along the line A-A of FIG. 1A.

FIG. 2 is an enlarged longitudinal cross-sectional view showing an example of a substrate according to a second embodiment of the present invention.

FIG. 3 is an enlarged longitudinal cross-sectional view showing an example of a substrate according to a third embodiment of the present invention.

FIG. 4 is an enlarged plan view showing an example of a substrate according to a fourth embodiment of the present invention.

FIG. 5 is an enlarged plan view showing an example of a substrate according to a fifth embodiment of the present invention.

FIG. 6A is a longitudinal cross-sectional view showing an example of a production process of a substrate in which no level difference is provided on the surface of the base material, according to the present invention.

FIG. 6B is a longitudinal cross-sectional view showing the example of the production process of the substrate in which no level difference is provided on the surface of the base material, according to the present invention.

FIG. 6C is a longitudinal cross-sectional view showing the example of the production process of the substrate in which no level difference is provided on the surface of the base material, according to the present invention.

FIG. 6D is a longitudinal cross-sectional view showing the example of the production process of the substrate in which no level difference is provided on the surface of the base material, according to the present invention.

FIG. 6E is a longitudinal cross-sectional view showing the example of the production process of the substrate in which no level difference is provided on the surface of the base material, according to the present invention.

FIG. 7A is a longitudinal cross-sectional view showing another example of the production process of the substrate in which no level difference is provided on the surface of the base material, according to the present invention.

FIG. 7B is a longitudinal cross-sectional view showing the another example of the production process of the substrate in which no level difference is provided on the surface of the base material, according to the present invention.

FIG. 7C is a longitudinal cross-sectional view showing the another example of the production process of the substrate in which no level difference is provided on the surface of the base material, according to the present invention.

FIG. 7D is a longitudinal cross-sectional view showing the another example of the production process of the substrate in which no level difference is provided on the surface of the base material, according to the present invention.

FIG. 8A is a longitudinal cross-sectional view showing an example of a production process of a substrate in which a level difference is provided on the surface of the base material, according to the present invention.

FIG. 8B is a longitudinal cross-sectional view showing the example of the production process of the substrate in which a level difference is provided on the surface of the base material, according to the present invention.

FIG. 8C is a longitudinal cross-sectional view showing the example of the production process of the substrate in which a level difference is provided on the surface of the base material, according to the present invention.

FIG. 8D is a longitudinal cross-sectional view showing the example of the production process of the substrate in which a level difference is provided on the surface of the base material, according to the present invention.

FIG. 8E is a longitudinal cross-sectional view showing the example of the production process of the substrate in which a level difference is provided on the surface of the base material, according to the present invention.

FIG. 8F is a longitudinal cross-sectional view showing the example of the production process of the substrate in which a level difference is provided on the surface of the base material, according to the present invention.

FIG. 8G is a longitudinal cross-sectional view showing the example of the production process of the substrate in which a level difference is provided on the surface of the base material, according to the present invention.

FIG. 9A is a longitudinal cross-sectional view showing another example of the production process of the substrate in which a level difference is provided on the surface of the base material, according to the present invention.

FIG. 9B is a longitudinal cross-sectional view showing the another example of the production process of the substrate in which a level difference is provided on the surface of the base material, according to the present invention.

FIG. 9C is a longitudinal cross-sectional view showing the another example of the production process of the substrate in which a level difference is provided on the surface of the base material, according to the present invention.

FIG. 9D is a longitudinal cross-sectional view showing the another example of the production process of the substrate in which a level difference is provided on the surface of the base material, according to the present invention.

FIG. 9E is a longitudinal cross-sectional view showing the another example of the production process of the substrate in which a level difference is provided on the surface of the base material, according to the present invention.

### BRIEF DESCRIPTION OF THE REFERENCE SYMBOLS

1′, 1″, 2, 3, 4, 4′, 5, and 6: Substrate for cell adhesion or culture
11, 11′, 11″, 21, 31, 51, and 61: Base material
12, 22, 32, 42, 52, and 62: Non-cell adhesion layer
121, 421, and 421′: Non-cell adhesion surface
13, 23, 33, 43, 53, and 63: Cell adhesion layer
131, 431, and 431′: Cell adhesion surface
431a, 431b, 431c, and 431d: Cell allocation zone
431e, 431f, 431g, and 431h: Cell connection zone
24, 34, 54, and 64: Photosensitive resin
111, 111′, 111″, 211, 311, 511, and 611: Exposed surface

### BEST MODE FOR CARRYING OUT THE INVENTION

### Substrate for Cell Adhesion or Culture

### First Embodiment

A substrate for cell adhesion or culture according to the present invention is capable of adhering and arranging cells on the surface thereof, as well as being suitable for culturing the thus adhered and arranged cells.

In the present invention, cells to be adhered onto the substrate are not specifically limited and can be appropriately selected according to the purpose. Moreover, kinds of cells which can be proliferated by culture are also preferred. Of these, animal cells are preferred, and particularly preferred cells can be specifically exemplified by neuroblasts, cardiomyocytes, hepatocytes, and adipocytes. Cells having a differential ability, for example, embryonic stem cells (ES cells) and induced pluripotent stem cells (iPS cells) may also be used.

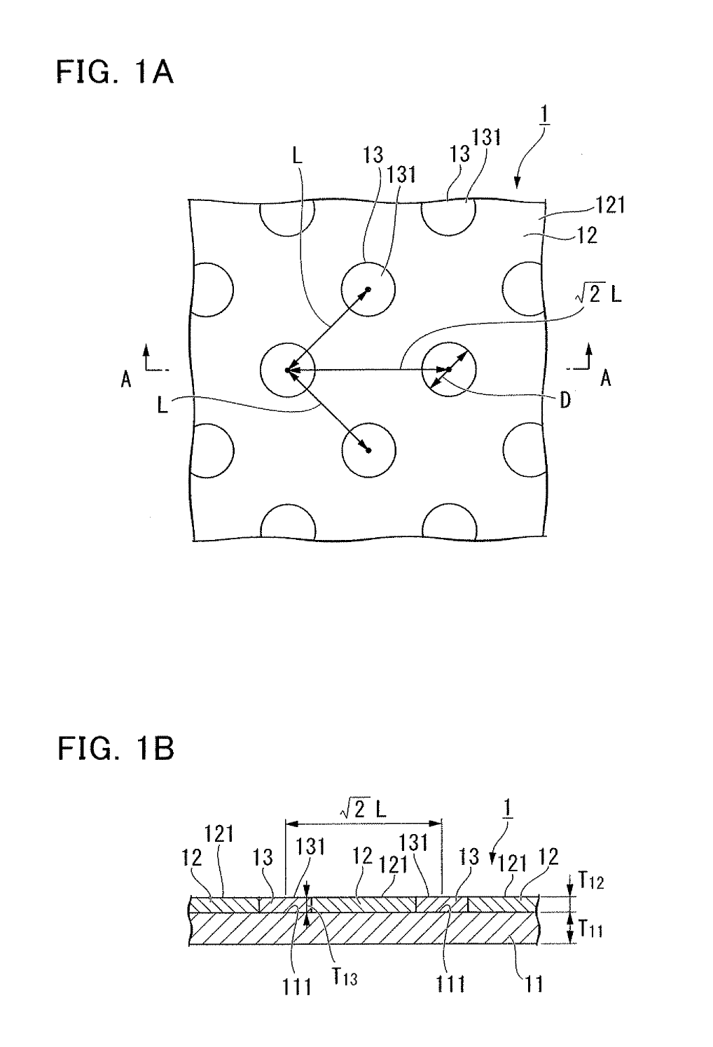

FIG. 1A and FIG. 1B show an example of a substrate for cell adhesion or culture according to a first embodiment of the present invention. FIG. 1A is an enlarged plan view and FIG. 1B is a longitudinal cross-sectional view taken along the line A-A of FIG. 1A.

In the substrate for cell adhesion or culture (hereunder, abbreviated as “substrate”) 1, a region other than predetermined regions of the surface of a base material 11 is covered with a non-cell adhesion layer (hereunder, abbreviated as “non-adhesion layer”) 12. Moreover, the predetermined region (hereunder, abbreviated as “exposed surface”) 111 of the surface of the base material 11 which is exposed without being covered by this non-adhesion layer 12, is covered with a cell adhesion layer (hereunder, abbreviated as “adhesion layer”) 13.

The material of the base material 11 may be the same as that for use in conventional biochips and the like.

Specifically, examples thereof can include a glass, a resin, a metal, and a ceramic. Of these, particularly preferred is a glass.

The adhesion layer 13 contains a substance having an adhesiveness to cells (hereunder, abbreviated as “adhesive substance”), and the exposed surface thereof (hereunder, abbreviated as “adhesion surface”) 131 is capable of adhering cells either at the time of culture or at any other time. Here, the term “adhesive substance” refers to a substance which has an affinity to cells, and examples thereof can include a peptide, a protein, and a glycoprotein. Of these, preferred adhesive substances can be exemplified by components of the extracellular matrix, which can be specifically exemplified by a collagen, a proteoglycan, a fibronectin, and a laminine. In addition, other preferred examples can include cytokine and poly-L-lysine. These adhesive substances are usually immobilized by adsorption when applied to the surface of the base material 11.

The adhesion layer 13 may contain either one type or a plurality of types of adhesive substances. If a plurality of types of adhesive substances is used, the combination, the ratio, and the like of them can be appropriately selected according to the purpose.

In addition, the adhesion layer 13 may also contain any constituent other than the adhesive substance, as long as the effect of the present invention is not impaired.

The non-adhesion layer 12 contains a substance having non-adhesiveness to cells (hereunder, abbreviated as “non-adhesive substance”), and the exposed surface thereof (hereunder, abbreviated as “non-adhesion surface”) 121 prevents cells from adhering thereto either at the time of culture or at any other time. The non-adhesion layer 12 has a light transmission characteristic.

Here, the term “light transmission characteristic” refers to a “property to transmit at least visible light” and preferably has a transparency equivalent to that of a transparent glass and silicon dioxide (SiO₂).

The refractive index of the non-adhesion layer 12 is preferably equivalent to that of a transparent glass and silicon dioxide (SiO₂), and preferably within 1.4 to 1.6.

By providing the non-adhesion layer 12 with such preferable physical properties, noise can be effectively reduced when cells and substances acting on these cells are to be optically detected, and therefore analyses can be performed with higher sensitivity.

The non-adhesion layer 12 can be specifically exemplified by one having a light transmission characteristic and a liquid repellency. Here, the term “liquid repellency” refers to a “repellency to water” or a “repellency to oil”. Such a non-adhesion layer 12 can be more specifically exemplified by layers which contain a fluoride-containing compound having a light transmission characteristic and a liquid repellency as a non-adhesive substance.

Preferred examples of this fluoride-containing compound can include compounds represented by the following general formula (1) (hereunder, abbreviated as the “compound (1)”):

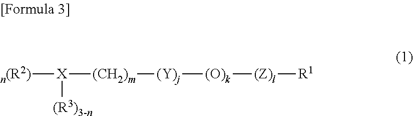

(In the formula, R¹ represents a linear or branched perfluoroalkyl group or perfluoroalkylether group of 1 to 16 carbon atoms; R² represents a hydroxyl group, or an atom or group which can be substituted by a hydroxyl group; R³ represents a hydrogen atom or a monovalent hydrocarbon group; X represents a silicon atom or a phosphorus atom; Y is a group represented by —NH—C(═O)— or a carbonyl group; Z represents an ethyleneoxy group having a hydrogen atom substituted by an alkyl group or alkyloxyalkyl group, one or more hydrogen atom(s) of which can be substituted by fluorine atom(s); j and k represent 0 or 1; 1 and m represent an integer of 0 or larger; n represents 1, 2, or 3; if n represents 2 or 3, n pieces of R² may be the same as or different from each other; if n represents 1, two pieces of R³ may be the same as or different from each other; and if 1 represents an integer of 2 or greater, 1 pieces of Z may be the same as or different from each other).

R¹ represents a linear or branched perfluoroalkyl group or perfluoroalkylether group of 1 to 16 carbon atoms. Here, the term “perfluoroalkyl group” refers to an “alkyl group in which all hydrogen atoms are substituted by fluorine atoms”. In addition, the term “perfluoroalkylether group” refers to a “monovalent group formed by bonding an oxygen atom to the above-mentioned perfluoroalkyl group”.

The above-mentioned alkyl in R¹ can be exemplified by methyl, ethyl, n-propyl, isopropyl, n-butyl, isobutyl, sec-butyl, tert-butyl, pentyl, hexyl, heptyl, octyl, nonyl, decyl, undecyl, dodecyl, tridecyl, tetradecyl, pentadecyl, and hexadecyl group.

The number of carbons of R¹ is preferably 3 to 12, and more preferably 6 to 10.

In addition, R¹ preferably takes a linear form, and is preferably a perfluoroalkyl group.

Preferable examples of the atom which can be substituted by a hydroxyl group of R² can include halogen atoms such as a fluorine atom, a chlorine atom, a bromine atom, and an iodine atom. Of these, a chlorine atom is particularly preferred.

In addition, preferable examples of the group of R² which can be substituted by a hydroxyl group can include an alkoxy group of 1 to 6 carbon atoms, an aryloxy group, an aralkyloxy group, and an acyloxy group.

The alkoxy group of 1 to 6 carbon atoms can be exemplified by a methoxy group, an ethoxy group, an n-propoxy group, an isopropoxy group, an n-butoxy group, an isobutoxy group, a sec-butoxy group, a tert-butoxy group, a pentoxy group, and a hexyloxy group.

The aryloxy group can be exemplified by a phenoxy group and a naphthoxy group.

The aralkyloxy group can be exemplified by a benzyloxy group and a phenethyloxy group.

The acyloxy group can be exemplified by an acetoxy group, a propionyloxy group, a butyryloxy group, a valeryloxy group, a pivaloyloxy group, and a benzoyloxy group.

Of these, more preferred are a chlorine atom, a methoxy group, and an ethoxy group, and a particularly preferred is a chlorine atom.

The monovalent hydrocarbon group of R³ is not specifically limited, and it may take either a chain structure or a cyclic structure and may be either saturated or unsaturated. If it takes a chain structure, the structure may be either linear or branched. If it takes a cyclic structure, the structure may be either monocyclic or polycyclic.

Of these, preferred are an alkyl group of 1 to 6 carbon atoms, an aryl group, and an aralkyl group.

The alkyl group of 1 to 6 carbon atoms can be exemplified by a methyl group, an ethyl group, an n-propyl group, an isopropyl group, an n-butyl group, an isobutyl group, a sec-butyl group, a tert-butyl group, a pentyl group, and a hexyl group.

The aryl group can be exemplified by a phenyl group and a naphthyl group.

The aralkyl group can be exemplified by a benzyl group and a phenethyl group.

Of these, more preferred are a methyl group, an ethyl group, an n-propyl group, and an isopropyl group, and a particularly preferred is a methyl group.

X represents a silicon atom or a phosphorus atom. For example, a silicon atom is preferred to facilitate the immobilization of the compound (1) to a glass substrate. In addition, a phosphorus atom is preferred to facilitate the immobilization of the compound (1) to a metal substrate.

Y is a group represented by —NH—C(═O)— or a carbonyl group. That is, the adjacent methylene group and oxygen atom are bound as follows.

“—(CH₂)ₘ—(NH—C(═O))ⱼ—(O)ₖ—”
“—(CH₂)ₘ—(C(═O))ⱼ—(O)ₖ—”

Z represents an ethyleneoxy group having a hydrogen atom substituted by an alkyl group or alkyloxyalkyl group, one or more hydrogen atom(s) of which can be substituted by fluorine atom(s). The oxygen atom at the end of the ethyleneoxy group is bound to the adjacent R¹.

The number of hydrogen atom(s) which can be substituted in the ethyleneoxy group is preferably one. The alkyl group or alkyloxyalkyl group, one or more hydrogen atom(s) of which can be substituted by fluorine atom(s), preferably takes a linear form or a branched form, and more preferably a linear form. In addition, the number of carbon atoms is preferably 1 to 16.

The symbols j and k represent 0 or 1, and particularly preferably 0.

The symbol l represents an integer of 0 or larger, and particularly preferably 0.

The symbol m represents an integer of 0 or larger, preferably 0 to 3, more preferably 1 or 2, and particularly preferably 2.

The symbol n represents 1, 2, or 3.

More preferred examples of the compound (1) are compounds represented by the following general formula (2):

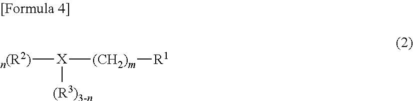

(In the formula, R¹, R², R³, X, m, and n have the same meanings as those mentioned above).

In addition, more preferred examples can also include compounds in which Z is represented by “—(CHR⁴—CHR⁵—O)_{l′}—(CHR⁶—CHR⁷—O)_{l″}” in the general formula (1).

Here, either one of R⁴ and R⁵ represents a hydrogen atom, and the other one of them represents a group in which one hydrogen atom of a methyl group is substituted by a linear or branched fluoroalkyl group of 1 to 16 carbon atoms or a fluoroalkylether group. Here, the term “fluoroalkylether group” refers to a “monovalent group formed by bonding an oxygen atom to the above-mentioned linear or branched fluoroalkyl group of 1 to 16 carbon atoms”.

Here, either one of R⁶ and R⁷ represents a hydrogen atom, and the other one of them represents a linear or branched alkyl group of 1 to 16 carbon atoms or an alkylether group. Here, the term “alkylether group” refers to a “monovalent group formed by bonding an oxygen atom to the above-mentioned linear or branched alkyl group of 1 to 16 carbon atoms”.

The symbol l′ represents an integer of 2 or greater, preferably 2 to 20, and more preferably 5 to 10.

The symbol l″ represents an integer of 0 or greater, preferably 0 to 20, and more preferably 0.

The fluoroalkyl group which substitutes for one hydrogen atom of the methyl group in R⁴ or R⁵ can be exemplified by CF₃—, CHF₂—, CClF₂—, C₂F₅—, CHF₂CF₂—, CClF₂CF₂—, (CF₃)₂CF—, CF₃CF₂CF₂—, (CHF₂)₂CF—, (CF₃)(CHF₂)CF—, (CF₃)₂CFCF₂CF₂—, (CF₃)₂CF(CF₂CF₂)₂—, CF₃(CF₂)₃—, CF₃(CF₂)₅—, CF₃(CF₂)₇—, CF₃(CF₂)₉—, CF₃(CF₂)₁₁—, CF₃(CF₂)₁₃—, and CF₃(CF₂)₁₅—.

Moreover, the fluoroalkylether group which substitutes for one hydrogen atom of the methyl group in R⁴ or R⁵ can be exemplified by monovalent groups formed by bonding an oxygen atom to these fluoroalkyl groups.

The alkyl group in R⁶ and R⁷ can be exemplified by methyl, ethyl, n-propyl, isopropyl, n-butyl, isobutyl, sec-butyl, tert-butyl, pentyl, hexyl, heptyl, octyl, nonyl, decyl, undecyl, dodecyl, tridecyl, tetradecyl, pentadecyl, and hexadecyl group.

Moreover, the alkylether group in R⁶ and R⁷ can be exemplified by monovalent groups formed by bonding an oxygen atom to these alkyl groups.

In the present invention, particularly preferred compounds (1) are those represented by the general formula (2).

Commercial products may be used for the compound (1). For example, CYTOP series (trade name, manufactured by ASAHI GLASS COMPANY), Megafac (trade name, manufactured by DIC Corporation), DIC Guard (trade name, manufactured by DIC Corporation), FPX-30G (trade name, manufactured by JSR Corporation), Novec EGC-1720 (trade name, manufactured by Sumitomo 3M Co., Ltd.), and Patinal series (substances WR1, WR2, and WR3) (trade names, manufactured by Merck Co., Ltd.), can be enumerated.

In the non-adhesion layer 12, the compound (1) is oriented so that its silicon atom or phosphorus atom can be bound to a specific atom or functional group on the base material (for example, oxygen atom in the case of a glass substrate) and R¹ can be exposed outward from the base material. It is assumed that such an orientation of R¹ can produce the non-adhesiveness to cells. Accordingly, it is important that R¹ is either a perfluoroalkyl group or a perfluoroalkylether group for the substrate 1 of the present invention to achieve a higher non-adhesion effect.

Either one type or a plurality of types of the non-adhesive substances may be used. If a plurality of types of the non-adhesive substances is used, the combination, the ratio, and the like of them can be appropriately selected according to the purpose.

The non-adhesion layer 12 may also contain any constituent other than the non-adhesive substance, as long as the effect of the present invention is not impaired.

The non-adhesion layer 12 has excellent transparency, is suitable for making a thin monolayer film, and has excellent processability for microfabrications including patterning and removal, as compared to conventional layers containing polytetrafluoroethylene and the like.

Because of such excellent transparency, noise can be greatly reduced when optically detect cells and substances acting on these cells. Therefore, analyses can be performed with higher sensitivity. Moreover, because of excellent processability for microfabrications, a large number of minute-sized adhesion surfaces can be formed on the substrate as will be described later, and the cell arrangement on the substrate 1 can be densified. Therefore, analyses can be performed with high efficiency.

The adhesion surface 131 is in an approximately circular shape. Adjacent adhesion surfaces 131 are arranged in an alignment at a fixed pitch so that the shortest center-to-center distance becomes L.

All of; the thickness T₁₁ of the substrate 11, the thickness T₁₂ of the non-adhesion layer 12, the diameter D of the adhesion surface 131, and the center-to-center distance L can be appropriately set in consideration of the size, the processability, and the usability of the substrate 1, and the like.

For example, in terms of the usability, the thickness T₁₁ is preferably 0.1 to 10 mm, and more preferably 0.3 to 1.5 mm In addition, in terms of the processability, the thickness T₁₂ is preferably 5 to 200 nm, and the thickness of the monomolecular layer formed from the above-mentioned compound (1) or the like is approximately equal to this.

On the other hand, the diameter D can be selected according to the purpose. For example, when cells are cultured on the adhesion surface 131, it is normally preferable that the diameter D is 2 to 500 μm, and more preferably 2 to 50 μm, although it depends on the cell type. A smaller diameter D is preferred as the number of the adhesion surfaces 131 on the substrate 1 can be increased.

The center-to-center distance L may be appropriately set in consideration of desired conditions such as the diameter D and the number of the adhesion surfaces 131 on the substrate 1.

The thickness of the adhesion layer 13 is not specifically limited.

In the present invention, as described above, the microfabrication of the non-adhesion layer 12 is possible, and the thickness T₁₂, the diameter D, and the center-to-center distance L, and the like can be set to be smaller than those of conventional substrates. Furthermore, the preciseness of fabrication is not impaired.

The number of the adhesion surfaces 131 can be optionally selected according to the purpose. Although only one adhesion surface 131 may be arranged, the number of the adhesion surfaces 131 is preferably larger, as the substrate 1 can be used for various analyses.

The manner of the arrangement of the adhesion layer is not limited to the arrangement shown herein. For example, here, the adhesion surface 131 is in an approximately circular shape in the illustrated case, although it may be in any other shape such as a polygonal shape and an oval shape. Of these, preferred is an approximately quadrangle shape, and more preferred is an approximately square shape. Moreover, all of the plurality of the adhesion layers 13 may not have approximately the same shape and size, and some or all of them may be different. Furthermore, the exposed surface 111 and the adhesion surface 131 have approximately the same shapes (the adhesion layer 13 is in an approximately columnar shape) in the illustrated case, although they may have different shapes or similar shapes. The shapes of the exposed surface and the adhesion surface can be adjusted, for example, by adjusting the patterning shape of the non-adhesion layer as will be described later.

Moreover, the thickness T₁₃ of the adhesion layer 13 and the thickness T₁₂ of the non-adhesion layer 12 are equivalent to each other, and the adhesion surface 131 and the non-adhesion surface 121 exist in approximately the same plane in the illustrated case, although a level difference may be provided between the adhesion surface 131 and the non-adhesion surface 121.

Furthermore, all of the thicknesses T₁₃ of the adhesion layers 13 and the thickness T₁₂ of the non-adhesion layer 12 may be the same as shown here, or some or all of them may be different from each other.

### Second Embodiment

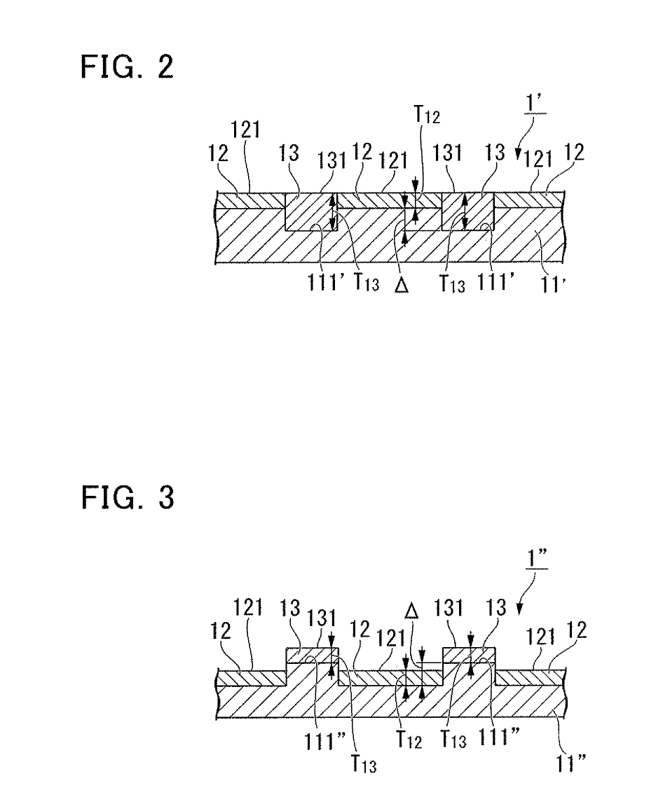

FIG. 2 is an enlarged longitudinal cross-sectional view showing an example of a substrate according to a second embodiment of the present invention.

In the substrate 1′ of this embodiment, the exposed surface 111′ is provided at a lower level by the difference of Δ than other surfaces, by which a level difference is created in the surface of the base material 11′. Here, the thickness T₁₃ of the adhesion layer 13 is made thicker than the thickness T₁₂ of the non-adhesion layer 12 so that the adhesion surface 131 and the non-adhesion surface 121 can exist in approximately the same plane. However, for example, the thickness T₁₃ may be made thinner than this so that another level difference can be additionally created between the adhesion surface 131 and the non-adhesion surface 121.

The height Δ of the level difference is preferably 0.01 to 100 μm, more preferably 0.5 to 70 μm, and particularly preferably 1 to 60 μm. By setting the height within such a preferable range, the handleability of the substrate can be improved and such a substrate can be readily produced as will be described later.

Moreover, all of the exposed surfaces 111′ have the same level difference Δ in the illustrated case, although some or all of them may be located at different levels.

This embodiment is the same as the first embodiment in other points except for the above-mentioned point.

By providing the level difference in the surface of the base material 11 in this way, the adhesion surface 131 and the non-adhesion surface 121 can be readily and visually distinguished, and thus the handleability of the substrate can be improved.

For example, in the present invention, the non-adhesion layer has excellent transparency, and has an equivalent refractive index to that of a glass. For this reason, it is difficult to visually distinguish the non-adhesion layer from a glass serving as the base material, and it is difficult to visually check the pattern of the non-adhesion surface during the production process of the substrate. In addition, it is also difficult to visually check the pattern of the adhesion layer on the substrate if the adhesion layer similarly has a high transparency. In this case, for example, when specific cells are arranged in a specific region on the adhesion surface, particularly when only a single cell is arranged, usually optical tweezers, a manipulator, or the like is used. In this case however, it may be sometimes difficult to arrange the cell(s) in the desired region of the adhesion surface. However, once a level difference is provided between the region covered by the adhesion layer and the region covered by the non-adhesion layer on the base material, it becomes easy to visually distinguish the adhesion surface and the non-adhesion surface, and thus the cell(s) can be readily arranged.

### Third Embodiment

FIG. 3 is an enlarged longitudinal cross-sectional view showing an example of a substrate according to a third embodiment of the present invention.

In the substrate 1″ of this embodiment, the exposed surface 111″ is provided at a higher level by the difference of Δ than other surfaces, by which a level difference is created in the surface of the base material 11″. Here, the level difference is created so that the adhesion surface 131 can be positioned higher than the non-adhesion surface 121. However, the level difference may also be created so that the adhesion surface 131 can be positioned lower than the non-adhesion surface 121, or so that the adhesion surface 131 and the non-adhesion surface 121 can exist in approximately the same plane.

The height Δ of the level difference is the same as that of the second embodiment.

This embodiment is the same as the second embodiment in other points except for the above-mentioned point.

By providing the level difference in this way, similarly to the second embodiment, the adhesion surface 131 and the non-adhesion surface 121 can be readily and visually distinguished, and thus the handleability of the substrate can be improved.

### Fourth Embodiment

Regarding all substrates according to the first to third embodiments, a plurality of adhesion surfaces, if any, are separately provided apart from each other and enclosed by the non-adhesion surface. The present invention is not to be limited to such a configuration, and may take another configuration in which some or all of the plurality of individual adhesion surfaces are connected to each other by the adhesion surface.

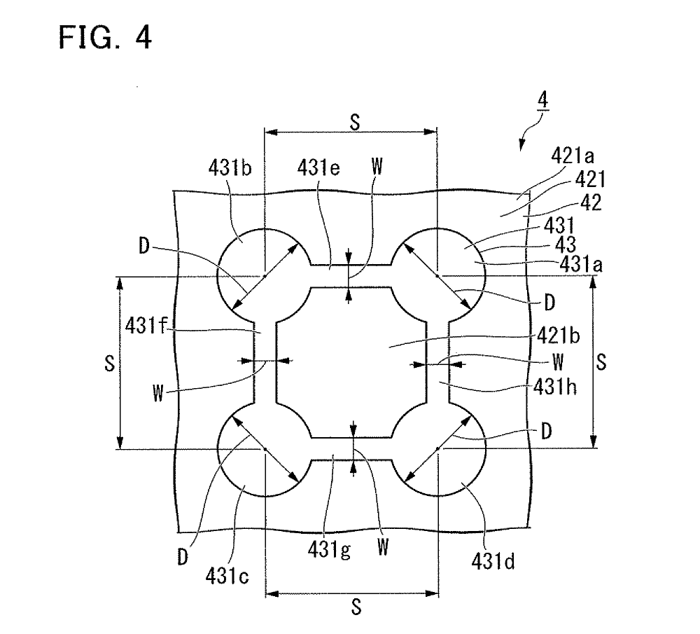

FIG. 4 is an enlarged plan view illustrating such a substrate according to a fourth embodiment of the present invention.

The substrate 4 of this embodiment is suitable for analyzing the function of an organized assembly of a plural number of cells in the case where the main action on the substrate is to connect these cells to each other rather than culturing them. Specifically, an adhesion surface 431 is provided on the substrate 4. The adhesion surface 431 comprises four cell allocation zones (hereunder, abbreviated as “allocation zones”) for arranging and adhering cells onto the substrate and four cell connection zones (hereunder, abbreviated as “connection zones”) for connecting these allocation zones.

More specifically, there are provided a first allocation zone 431a, a second allocation zone 431b, a third allocation zone 431c, and a fourth allocation zone 431d, and there are further provided a first connection zone 431e for connecting the first allocation zone 431a and the second allocation zone 431b, a second connection zone 431f for connecting the second allocation zone 431b and the third allocation zone 431c, a third connection zone 431g for connecting the third allocation zone 431c and the fourth allocation zone 431d, and a fourth connection zone 431h for connecting the fourth allocation zone 431d and the first allocation zone 431a.

The first allocation zone 431a to the fourth allocation zone 431d are sites for arranging and adhering cells as the target of analysis, while the first connection zone 431e to the fourth connection zone 431h are sites for connecting cells in the respective allocation zones to each other. All sites are formed of an adhesion surface.

Every one of the first allocation zone 431a to the fourth allocation zone 431d is in an approximately circular shape of a diameter D. Moreover, every one of the first connection zone 431e to the fourth connection zone 431h is in a belt-like shape having a width W. It is preferable that the arrangement of the respective connection zones is taken so that the respective allocation zones serving as the target of connection can be connected by the shortest distance. In FIG. 4, the connection zones are arranged so as to overlay the lines linking the approximately central points of the allocation zones. The arrangement of the respective allocation zones is taken so that their approximately central points are positioned at respective apexes of a square, and the connection distance S is equivalent to the length of each side line of the square.

By so doing, in this embodiment, the adhesion surface 431 divides the non-adhesion surface 421 into a first non-adhesion surface 421a enclosing the adhesion surface 431 and a second non-adhesion surface 421b enclosed by the adhesion surface 431.

The dimension of each allocation zone can be appropriately adjusted according to the type and the number of cells to be arranged, or any other purpose. For example, considering that the number of cells to be arranged and adhered is one per each allocation zone and these cells are not to be cultured, it is normally preferable that the dimension of each allocation zone is 2.0×10⁻¹¹ to 4.0×10⁻⁸ m², and more preferably 1.8×10⁻¹⁰ to 2.3×10⁻⁸ m². In order to set such a preferable dimension, the diameter D can be adjusted to match with this dimension, preferably at 5 to 225 μm, and more preferably 15 to 150 μm.

The width W and the connection distance S can also be appropriately adjusted according to the type and the number of cells to be arranged, or any other purpose. Similarly to the above-mentioned case, considering that the number of cells to be arranged and adhered is one per each allocation zone and these cells are not to be cultured, it is normally preferable that the width W is 1 to 30 μm, and more preferably 3 to 20 μm. The connection distance S is preferably adjusted according to the diameter D, preferably at D+5 to D+100 μm, and more preferably at D+7 to D+70 μm.

In this embodiment, the number of the adhesion surfaces 431 can be appropriately selected according to the purpose, and may be either singular or plural without any specific limitations.

In the present invention, the manner of the arrangement of the adhesion layer is not limited to the arrangement shown herein as long as an allocation zone and a connection zone are provided, and as long as the effect of the present invention is not impaired. For example, the allocation zone may take any shape other than the approximately circular shape, as long as cells can be arranged and adhered thereto, and it is preferably a non-slender shape such as a polygonal shape or an oval shape. Of these, preferred is an approximately quadrangle shape, and more preferred is an approximately square shape. The connection zone may take any slender shape. Moreover, the connection zones may also be arranged to overlay diagonal lines of the square in addition to or without the respective side lines of the square, or may take any other arrangement. In addition, the shapes of the respective allocation zones are all the same in the illustrated case. However, all of them do not have to be the same and some or all of them may be different. The same thing can be said regarding the sizes of the respective allocation zones, and the shapes and the sizes of the respective connection zones.

Furthermore, the numbers of the allocation zones and the connection zones can also be appropriately selected according to the purpose.

This embodiment is the same as the first to the third embodiments in other points except for the above-mentioned point.

### Fifth Embodiment

FIG. 5 is an enlarged plan view showing an example of a substrate according to a fifth embodiment of the present invention.

In the substrate 4′ of this embodiment, the allocation zone and the connection zone are similar to those of the substrate 4 mentioned above, but the numbers of these allocation zones and connection zones of an adhesion surface 431′ and the shape of a non-adhesion surface 421′ are different from those of the substrate 4. The other points are the same as those of the fourth embodiment.

The surface of the substrate of the present invention except for the adhesion surface is covered by the non-adhesion surface. Therefore, upon the application of cells onto the substrate for analyses, even if cells are adhered to a region other than the adhesion surface, these cells can be readily removed.

In addition, as will be described later, upon the production of the substrate, the adhesive substance can also be prevented from being adsorbed to a region other than the exposed surface of the base material. Therefore, impurities, which can be a cause of noise, can be prevented from being adsorbed to the region other than the adhesion surface of the substrate. Furthermore, since the non-adhesion layer has excellent transparency, noise can be greatly reduced when optically detects cells and substances acting on these cells. Accordingly, at the time of analysis, the S/N ratio can be greatly improved as compared to conventional substrates. Therefore, analyses can be performed with higher accuracy.

In addition, the non-adhesion layer is suitable for making a thin monolayer film, and has excellent processability for microfabrication. Accordingly, a large number of minute-sized adhesion surfaces can be formed, and thus the cell arrangement can be densified. Therefore, analyses can be performed with high efficiency.

With use of the substrate of the present invention, cells can be handled per each individual cell, and the function thereof can be analyzed with high accuracy. Thus, various analyses and material productions utilizing the function of cells can also be performed with high efficiency. In addition, these analyses and material productions can be performed by the same methods as methods in which a conventional biochip or the like is used, except for employing the substrate of the present invention.

### Method for Producing Substrate for Cell Adhesion or Culture

The above-mentioned substrate of the present invention can be produced in the following manner.

### (Production Method 1)

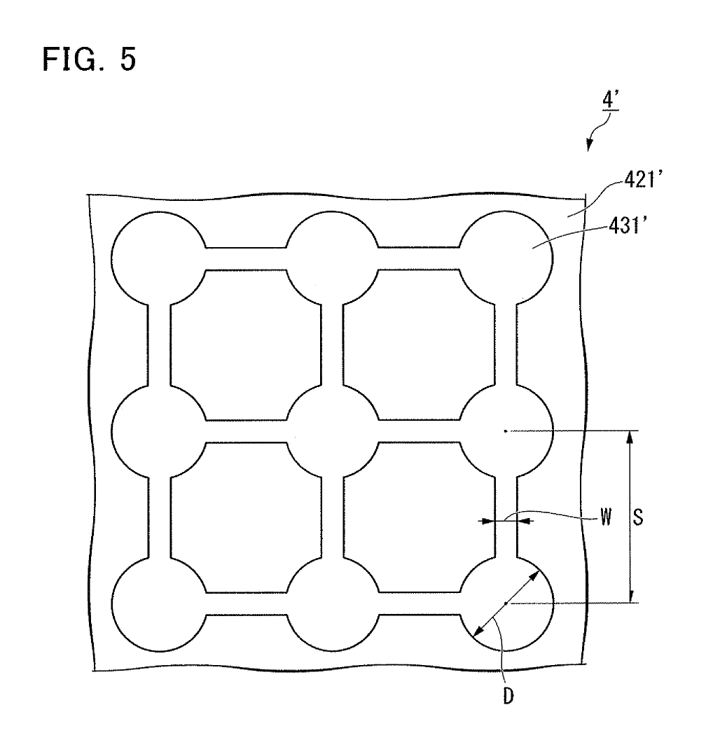

FIGS. 6A to 6E are longitudinal cross-sectional views showing an example of the production process of a substrate in which no level difference is provided on the surface of the base material, similarly to the substrate according to first embodiment of the present invention.

First, as shown in FIG. 6A, the entire surface of one side of a base material 21 is covered by a non-adhesion layer 22. Examples of the covering method include a dip coating method, a vacuum evaporation method, a CVD (chemical vapor deposition) method, and a plasma polymerization method. For example, in the case of a dip coating method, a coating treatment solution containing a non-adhesive substance such as the compound (1) is prepared and applied to the entire surface of the one side of the base material 21. After the application, the solvent component in the treatment solution is preferably removed by drying. The thickness of the non-adhesion layer 22 can be adjusted by the size and the quantity of molecules of the non-adhesive substance.

Next, the base material 21 covered by the non-adhesion layer 22 is preferably washed and dried. The washing can be carried out with use of an alcohol such as methanol and ethanol, or may also be carried out with use of a fluorine-based solvent if the coating treatment solution contains a fluorine-based solvent.

Then, as shown in FIG. 6B, a pattern is made on the non-adhesion layer 22 with the photosensitive resin 24 by photolithography. The photolithography may be a known method.

Thereafter, the non-adhesion layer 22 is etched by using the pattern of the photosensitive resin 24 as a mask. The etching method may be any method, although dry etching is preferred. More specifically preferred is etching with use of a plasma generated from a species of gas selected from the group consisting of oxygen, argon, and chlorine.

The non-adhesion layer 22 which is not masked with the photosensitive resin 24 is removed by etching, by which as shown in FIG. 6C, a pattern of the non-adhesion layer 22 is made.

Next, the photosensitive resin 24 remaining on the base material 21 is all removed, by which the base material 21, the surface of which has a pattern of the non-adhesion layer 22 as shown in FIG. 6D, can be obtained.

Then, the exposed surface 211 of the base material 21, which has been exposed by removing the non-adhesion layer 22, is covered with the adhesion layer 23. In this case, it is preferable that, for example, a solution containing an adhesive substance is prepared and the solution is applied to the exposed surface 211, and then, as required, washed with a washing liquid and dried. The application of the solution onto the exposed surface 211 may also be performed by soaking the base material 21 formed with the exposed surface 211 in the solution.

The solvent component in the solution may be appropriately selected according to the type of the adhesive substance. For example, as most of adhesive substances are water-soluble, it is preferable to use an aqueous solution such as a buffer solution. The solution may also contain a constituent other than the adhesive substance, if necessary. In addition, if it is to be dried, the drying is preferably performed at about room temperature so as not to deteriorate the adhesive substance.

By so doing, the substrate 2 having the exposed surface 211 covered with the adhesion layer 23 as shown in FIG. 6E is obtained. The thickness of the adhesion layer 23 can be adjusted by the size and the quantity of molecules of the adhesive substance.

In the present invention, upon the application of the solution onto the exposed surface 211, even if this solution is adhered to a region other than the exposed surface 211, the solution can be readily removed because the region is covered by the non-adhesion layer 22. Therefore, the adhesive substance can be prevented from adhering to the region other than the desired region.

### (Production Method 2)

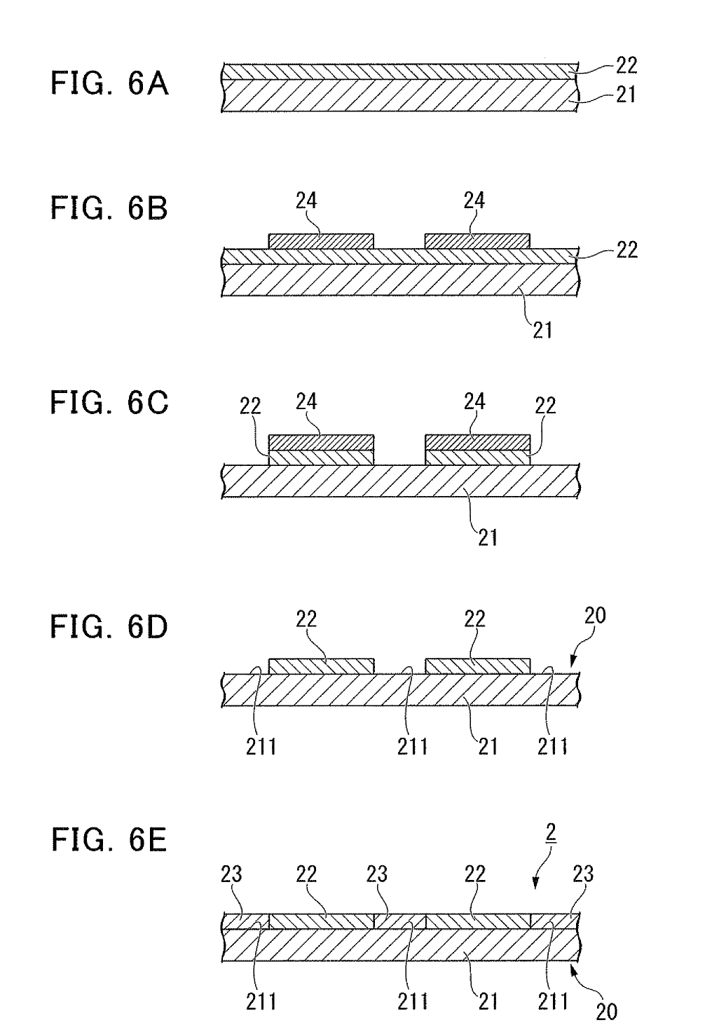

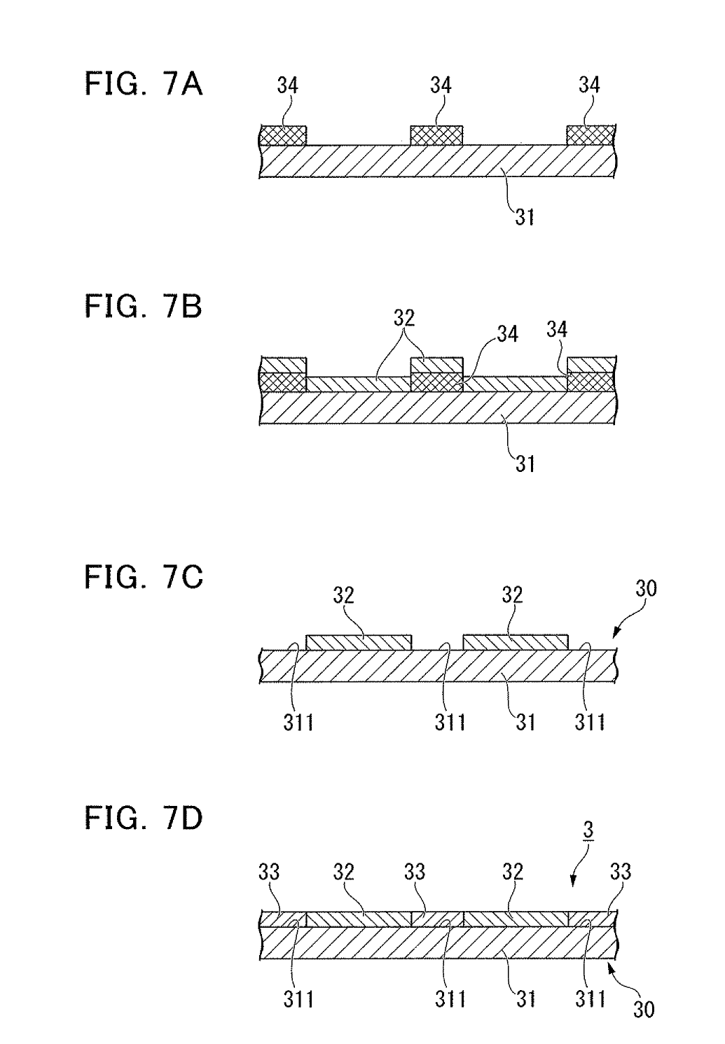

FIGS. 7A to 7D are longitudinal cross-sectional views showing another example of the production process of a substrate in which no level difference is provided on the surface of the base material.

First, as shown in FIG. 7A, patterning is performed to a photosensitive resin 34 provided on the surface of a base material 31 by photolithography.

Next, as shown in FIG. 7B, the photosensitive resin 34 and the surface of the exposed area of the base material 31 are covered with a non-adhesion layer 32. The covering method is not specifically limited, and may be the same as mentioned above.

Next, as shown in FIG. 7C, the non-adhesion layer 32 on the photosensitive resin 34 is removed together with the photosensitive resin 34 covered by this non-adhesion layer 32. By so doing, a pattern of the non-adhesion layer 32 can be made on the base material 31.

Then, the exposed surface 311 of the base material 31 is covered with an adhesion layer 33 in the same manner as described in the production method 1.

By so doing, the substrate 3 in which the exposed surface 311 is covered with the adhesion layer 33 as shown in FIG. 7D, can be obtained.

### (Production Method 3)

It is also possible to produce a substrate in which a level difference is provided in the surface of the base material, similarly to the substrate according to the second embodiment of the present invention, for example, by performing the respective processes of the production method described in FIGS. 6A to 6E, with use of a base material in which a desired level difference has been formed.

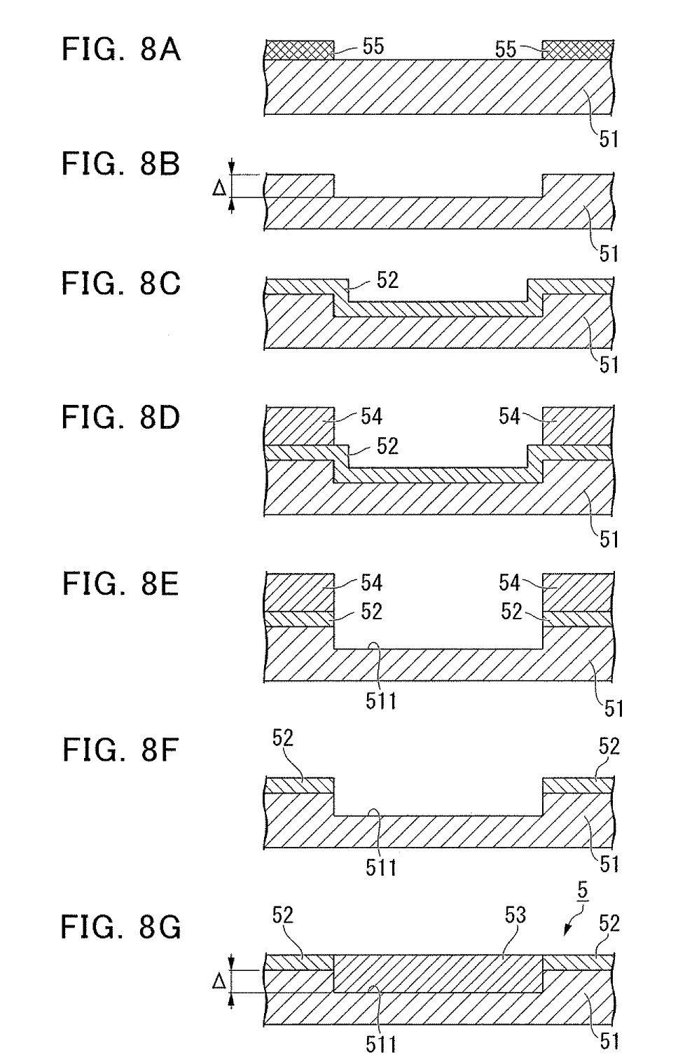

FIGS. 8A to 8G are longitudinal cross-sectional views showing an example of the production process of such a substrate.

First, patterning is performed to a photosensitive resin provided on the surface of a base material 51, a metal film 55 such as a chromium film is deposited thereon by sputtering, and then a pattern as shown in FIG. 8A is formed to the metal thin film 55 by a lift-off method.

Next, this base material 51 is soaked and held in an etching liquid at a suitable temperature to thereby perform wet etching by using the metal film 55 as a mask to etch a depth of Δ. Then, the metal film 55 is removed with another etching liquid to thereby form a level difference having the height Δ in the surface of the base material 51 as shown in FIG. 8B.

Thereafter, the respective process can be performed in the same manner as described in the production method 1 so as to form an adhesion layer on the etched surface of the base material 51.

Specifically, as shown in FIG. 8C, the surface of the base material 51 formed with the level difference is covered by the non-adhesion layer 52 in the same manner as mentioned above.

Next, as shown in FIG. 8D, patterning is performed to a photosensitive resin 54 provided on the non-adhesion layer 52. The photosensitive resin 54 is overlaid on the unetched surface of the base material 51 via the non-adhesion layer 52.

Thereafter, the non-adhesion layer 52 is etched by using the patterned photosensitive resin 54 as a mask, preferably in the same dry etching method as mentioned above.

The non-adhesion layer 52 which is not masked with the photosensitive resin 54 is removed by etching, by which as shown in FIG. 8E, a pattern of the non-adhesion layer 52 is made.

Next, the photosensitive resin 54 remaining on the base material 51 is all removed, by which the base material 51, the surface of which has a pattern of the non-adhesion layer 52 as shown in FIG. 8F, can be obtained.

Then, the exposed surface 511 of the base material 51, which has been exposed by removing the non-adhesion layer 52, is covered with an adhesion layer 53 in the same manner as mentioned above.

By so doing, as shown in FIG. 8G, the substrate 5 in which the level difference is provided in the surface of the base material 51, and the exposed surface 511 at a lower level of Δ is covered with the adhesion layer 53 is obtained.

### (Production Method 4)

It is also possible to produce a substrate in which a level difference is provided in the surface of the base material, similarly to the substrate according to the third embodiment of the present invention, for example, by performing the respective process of the production method described in FIGS. 7A to 7D, with use of a base material in which a desired level difference has been formed.

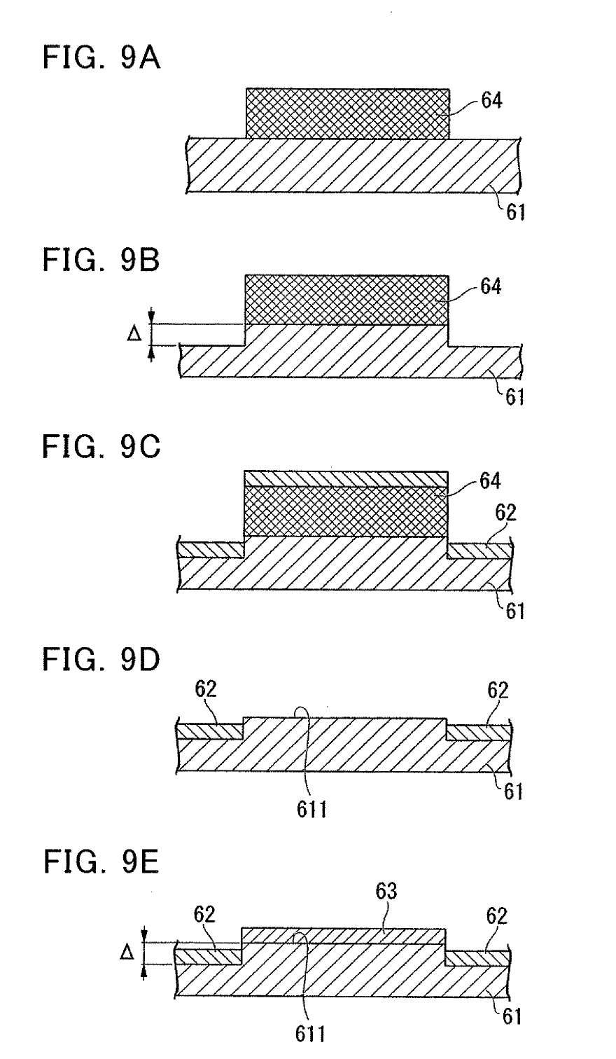

FIGS. 9A to 9E are longitudinal cross-sectional views showing an example of the production process of such a substrate.

First, as shown in FIG. 9A, patterning is performed to a photosensitive resin 64 which has a repellency to an etching liquid and provided on the surface of a base material 61, by photolithography or the like.

Next, this base material 61 is soaked and held in the etching liquid at a suitable temperature to thereby perform wet etching by using the photosensitive resin 64 as a mask. By so doing, as shown in FIG. 9B, a level difference having the height Δ is formed in the surface of the base material 61.

Thereafter, the respective process can be performed in the same manner as described in the method of FIGS. 7B to 7D so as to form an adhesion layer on the unetched surface of the base material 61.

Specifically, as shown in FIG. 9C, the photosensitive resin 64 and the surface of the exposed area of the base material 61 are covered with a non-adhesion layer 62 in the same manner as mentioned above.

Next, the non-adhesion layer 62 on the photosensitive resin 64 is removed together with the photosensitive resin 64 covered by this non-adhesion layer 62. By so doing, a pattern of the non-adhesion layer 62 can be made on the base material 61 as shown in FIG. 9D.

Then, the exposed surface 611 of the base material 61, which has been exposed by removing the non-adhesion layer 62, is covered with an adhesion layer 63 in the same manner as mentioned above.

By so doing, as shown in FIG. 9E, the base material 61 in which the level difference is provided in the surface of the base material 61, and the exposed surface 611 at a higher level of Δ is covered with the adhesion layer 63, is obtained.

### EXAMPLES

Hereunder is a more detailed description of the present invention with reference to specific examples. However, the present invention is not to be limited by the following examples.

### Example 1

A substrate was produced by the method described with FIGS. 6A to 6E.

That is, the entire surface of one side of a Pyrex (registered trademark) glass substrate (code 7059, manufactured by Corning International Co., Ltd.) (thickness; 1.1 mm) was dip-coated with a hexamethyldisiloxane solution which contained CF₃—(CF₂)₇—(CH₂)₂—Si(CH₃)₂Cl at a concentration of 0.02 mol/L. The substrate was dried overnight, then heated at 100° C. for 1 hour, and washed with isopropanol and ethanol. By so doing, a non-adhesion layer was formed.

Next, a dot pattern having a dot diameter of 30 μm was made with a photosensitive resin by photolithography. Etching with oxygen plasma was performed thereon to thereby remove predetermined regions of the non-adhesion layer.

Then, the photosensitive resin was removed. By so doing, a pattern of the non-adhesion layer was formed on the base material, and thereby the substrate formed with circular-shaped exposed surfaces having a diameter of 30 μm (shortest center-to-center distance of adjacent exposed surfaces; 80 μm) as shown in FIG. 1A and FIG. 1B was obtained.

Thereafter, a collagen solution was prepared by dissolving a Cell Matrix Type III (manufactured by Nitta Gelatin Inc.) in a dilute hydrochloric acid aqueous solution of pH 3 at a concentration of 0.3 mg/mL. The above-mentioned substrate formed with the exposed surfaces on the base material was soaked in this collagen solution to thereby apply the collagen onto the exposed surfaces, and then the substrate was washed with a dilute hydrochloric acid aqueous solution of pH 3, by which an adhesion layer was formed. By so doing, the substrate of the present invention was produced.

Neuroblasts PC-12 were cultured on the adhesion surfaces of the substrate. Even after a week culture, no adhesion of cells was found on the non-adhesion surfaces, confirming that the pattern of the cultured cells was not lost.

### Example 2

A substrate was produced by the method described with FIGS. 7A to 7D.

That is, a dot pattern having a dot diameter of 30 μm was made with a photosensitive resin by photolithography on a Pyrex (registered trademark) glass substrate (code 7740, manufactured by Corning International Co., Ltd.) (thickness; 0.5 mm)

Next, a non-adhesive substance was coated over the glass substrate and the photosensitive resin by a vacuum evaporation method with use of WR1 Patinal (trade name, manufactured by Merck Chemicals) as an evaporation source, to thereby form a non-adhesion layer. The temperature of the evaporation source was set at 360° C. to 450° C., and the evaporation coating was conducted at a degree of vacuum of 10⁻³ Pa for 30 seconds.

Then, the non-adhesion layer on the photosensitive resin was removed together with the photosensitive resin covered by this non-adhesion layer to thereby form exposed surfaces. Thereafter, a collagen solution was prepared by dissolving a Cell Matrix Type III (manufactured by Nitta Gelatin Inc.) in a dilute hydrochloric acid aqueous solution of pH 3 at a concentration of 0.3 mg/mL. The above-mentioned substrate formed with the exposed surfaces on the base material was soaked in this collagen solution to thereby apply the collagen onto the exposed surfaces, and then the substrate was washed with a dilute hydrochloric acid aqueous solution of pH 3, by which an adhesion layer was formed. By so doing, the substrate of the present invention was produced.

Cells were cultured in the same manner as that of example 1, by which similar results to those of example 1 were obtained.

### Example 3

A substrate having an adhesion surface in the same shape as that of FIG. 5 was produced by the method described with FIGS. 7A to 7D.

That is, a pattern was made with a photosensitive resin by photolithography on a Pyrex (registered trademark) glass substrate (code 7740, manufactured by Corning International Co., Ltd.) (thickness; 0.5 mm).

Next, the glass substrate and the photosensitive resin were dip-coated with a solution prepared by dissolving CF₃—(CF₂)₇—(CH₂)₂—SiCl₃ in a fluorine-based solvent Novec HFE (trade name, manufactured by Sumitomo 3M) at a concentration of 0.02 mol/L. The substrate was dried overnight, then heated at 100° C. for 1 hour, and washed with the Novec HFE. By so doing, a non-adhesion layer was formed.

Then, the non-adhesion layer on the photosensitive resin was removed together with the photosensitive resin covered by this non-adhesion layer using acetone to thereby form an exposed surface. The resultant exposed surface had the same shape as that of the adhesion surface shown in FIG. 5. The diameter D of each allocation zone was 35 μm, the connection distance S was 45 μm, and the width W of the connection zone was 15 μm.

Thereafter, a collagen solution was prepared by dissolving a Cell Matrix Type III (manufactured by Nitta Gelatin Inc.) in a dilute hydrochloric acid aqueous solution of pH 3 at a concentration of 0.3 mg/mL. The substrate formed with the exposed surface on the base material was soaked in this collagen solution to thereby apply the collagen onto the exposed surface, and then the substrate was washed with a dilute hydrochloric acid aqueous solution of pH3, by which an adhesion layer was formed. By so doing, the substrate of the present invention was produced.

Primary cultured cells of rat cardiomyocytes (manufactured by Hokkaido System Science Co., Ltd.) were seeded and cultured on the resultant substrate. As a result, it was confirmed that many cells were adhered on the allocation zones of the adhesion surface but not adhered on the non-adhesion surface. There were several types of allocation zones where, respectively, no cell, a single cell, and a plurality of cells, was/were arranged.

### Example 4

A substrate having an adhesion surface in the same shape as that of FIG. 5 was produced by the method described with FIGS. 9A to 9E.

That is, a pattern was made with a photosensitive resin, which had a repellency to a glass etching liquid containing hydrofluoric acid, by photolithography on a Pyrex (registered trademark) glass substrate (code 7059, manufactured by Corning International Co., Ltd.) (thickness; 0.7 mm).

Next, the substrate was soaked and held in the glass etching liquid (buffered hydrofluoric acid) at room temperature to thereby perform wet etching with use of the photosensitive resin as a mask, by which a level difference of 5 μm was created in the glass substrate.

Thereafter, a non-adhesion layer was formed in the same manner as that of example 3. Then, the non-adhesion layer on the photosensitive resin was removed together with the photosensitive resin covered by this non-adhesion layer, using NMP (N-methylpyrrolidone) to thereby form an exposed surface. The resultant exposed surface had the same shape and size as those of example 3.

Next, in the same manner as that of example 3, collagen was applied onto the exposed surface and the substrate was washed, by which an adhesion layer was formed. By so doing, the substrate of the present invention was produced. In the resultant substrate, because the level difference was provided in the glass substrate, the adhesion surface and the non-adhesion surface were visually distinguished.

Primary cultured cells of rat cardiomyocytes (manufactured by Hokkaido System Science Co., Ltd.) were seeded on the resultant substrate in the same manner as that of example 3. Subsequently, the cultured cells were arranged using optical tweezers by means of an infrared laser so that a single cell could be allocated and adhered per each allocation zone. At this time, as the allocation zone was able to be visually checked, it was easy to arrange these cultured cells. As a result, cultured cells were patterned on the adhesion surface without being allocated in the non-adhesion surface, and furthermore the cultured cells were connected each other in the connection zones and a synchronized heart beat of the cultured cells was observed.

### Example 5

A substrate having an adhesion surface in the same shape as that of FIG. 5 was produced by the method described with FIGS. 8A to 8G.

That is, a pattern was made with a photosensitive resin on a silica glass substrate (thickness; 0.5 mm), then a chromium (Cr) film was deposited thereon in a thickness of 100 nm by sputtering, and patterning is performed to the chromium film by a lift-off method.

Next, the substrate was soaked and held in an etching liquid (buffered hydrofluoric acid) at room temperature to thereby perform wet etching with use of the chromium film as a mask, by which a level difference of 50 μm was created in the silica glass substrate. Then, the chromium film was removed using a chromium etching liquid.

Then, the entire surface of the silica glass substrate which had been provided with the level difference was coated with a non-adhesive substance by a vacuum evaporation method with use of WR1 Patinal (trade name, manufactured by Merck Chemicals) as an evaporation source, to thereby form a non-adhesion layer. The temperature of the evaporation source was set at 360° C. to 450° C., and the evaporation coating was conducted at a degree of vacuum of 10⁻³ Pa for 30 seconds.

Next, patterning was performed to a photosensitive resin provided on the unetched surface of the silica glass substrate via the non-adhesion layer.

Thereafter, etching with oxygen plasma was performed by using the pattern of the photosensitive resin as a mask, by which a predetermined region of the non-adhesion layer was removed.

Then, the photosensitive resin was removed using NMP (N-methylpyrrolidone) to thereby form an exposed surface. The resultant exposed surface had the same shape and size as those of example 3.

Next, in the same manner as that of example 3, collagen was applied onto the exposed surface and the substrate was washed, by which an adhesion layer was formed. By so doing, the substrate of the present invention was produced. In the resultant substrate, the adhesion surface and the non-adhesion surface were able to be distinguished by eye similarly to example 4.

Primary cultured cells of rat cardiomyocytes (manufactured by Hokkaido System Science Co., Ltd.) were arranged so that a single cell could be allocated and adhered per each allocation zone on the resultant substrate in the same manner as that of example 4, by which similar results to those of example 4 were obtained.

### INDUSTRIAL APPLICABILITY

The present invention can be applied to the fields of researches of cell functions, bioassays and material productions which utilize cells, and the like.

1. A substrate for cell adhesion or culture, comprising:
a base material;
a cell adhesion layer arranged to cover a predetermined region on the base material; and
a non-cell adhesion layer arranged on the base material to cover a region other than said predetermined region; wherein
an exposed surface of said cell adhesion layer is a cell adhesion surface;
the non-cell adhesion layer has a light transmission characteristic; and
an exposed surface of the non-cell adhesion layer is a non-cell adhesion surface.

2. The substrate for cell adhesion or culture according to claim 1, wherein said non-cell adhesion layer contains a fluoride-containing compound represented by the following general formula (1): 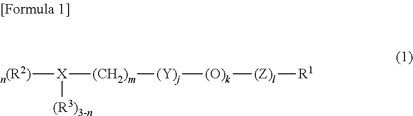
(wherein, R¹ represents a linear or branched perfluoroalkyl group or perfluoroalkylether group of 1 to 16 carbon atoms; R² represents a hydroxyl group, or an atom or group which can be substituted by a hydroxyl group; R³ represents a hydrogen atom or a monovalent hydrocarbon group; X represents a silicon atom or a phosphorus atom; Y is a group represented by —NH—C(═O)— or a carbonyl group; Z represents an ethyleneoxy group having a hydrogen atom substituted by an alkyl group or alkyloxyalkyl group, one or more hydrogen atom(s) of which can be substituted by fluorine atom(s); j and k represent 0 or 1; 1 and m represent an integer of 0 or larger; n represents 1, 2, or 3; if n represents 2 or 3, n pieces of R² may be the same as or different from each other; if n represents 1, two pieces of R³ may be the same as or different from each other; and if 1 represents an integer of 2 or larger, 1 pieces of Z may be the same as or different from each other).

3. The substrate for cell adhesion or culture according to claim 2, wherein the fluoride-containing compound represented by said general formula (1) is a compound represented by the following general formula (2): 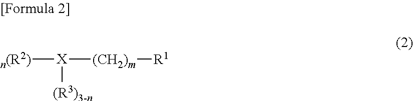
(wherein, R¹, R², R³, X, m, and n have the same meanings as those mentioned above).

4. The substrate for cell adhesion or culture according to claim 1, wherein a level difference is provided between said predetermined region covered by said cell adhesion layer and the region covered by said non-cell adhesion layer on said base material.

5. The substrate for cell adhesion or culture according to claim 4, wherein a height of said level difference is 0.01 to 100 μm.

6. The substrate for cell adhesion or culture according to claim 1, wherein a thickness of said non-cell adhesion layer is 5 to 200 nm.

7. The substrate for cell adhesion or culture according to claim 1, wherein a plurality of said cell adhesion surfaces having dimensions of 2.0×10⁻¹¹ to 4.0×10⁻⁸ m² are connected by said cell adhesion surface in a belt-like shape having a width of 1×10⁻⁶ to 3×10⁻⁵ m.

8. A method for producing the substrate for cell adhesion or culture according to claim 1, wherein the method comprises:
covering the surface of a base material with a non-cell adhesion layer;
removing a specific site of this non-cell adhesion layer to expose the surface of the base material; and
covering the thus exposed surface of the base material with a cell adhesion layer.

9. The method for producing a substrate for cell adhesion or culture according to claim 8, wherein a surface of the base material is exposed by:
covering the surface of the base material with a non-cell adhesion layer;
making a pattern on this non-cell adhesion layer with a photosensitive resin; and
removing the specific site of said non-cell adhesion layer by etching with use of the pattern of said photosensitive resin as a mask, and subsequently removing the photosensitive resin.

10. The method for producing a substrate for cell adhesion or culture according to claim 8, wherein a surface of the base material is exposed by:
making a pattern on the surface of the base material with a photosensitive resin;
covering said surface of the base material and said photosensitive resin with a non-cell adhesion layer; and
removing the non-cell adhesion layer on a site of the non-cell adhesion layer covering the photosensitive resin together with the photosensitive resin covered by this non-cell adhesion layer.

11. The method for producing a substrate for cell adhesion or culture according to claim 8, wherein the method further comprises:
forming a level difference in the surface of the base material; and
covering the surface of the base material with the non-cell adhesion layer after forming the level difference.

12. The method for producing a substrate for cell adhesion or culture according to claim 9, wherein the etching is performed with use of a plasma generated from a gas selected from the group consisting of oxygen, argon, and chlorine.
